(19) United States
(12) Patent Application Publication
(10) Pub. No.: US 2009/0191542 A1
(43) Pub. Date: Jul. 30, 2009

(54) Multi-variant cell indication technique

(75) Inventors: Geoffrey L. Hodge, Sutton, MA (US); Shari Brezinsky, Belmont, MA (US)

Correspondence Address: Xcellerex, Inc.;Attention: Jacqueline Arendt, 170 Locke Drive, Marlborough, MA, 01752, US

(73) Assignee: Xcellerex, Inc., Marlborough, MA (US)

(21) Appl. No.: 11/895,057

(22) Filed: Aug. 23, 2007

Related U.S. Application Data
(60) Provisional application No. 60/840,070, filed on Aug. 23, 2006.

(51) Int. Cl. C12Q 1/68; C12Q 1/02; C12Q 1/26; G01N 33/53; C12N 5/06; G06Q 90/00

(52) U.S. Cl. 435/6; 435/29; 435/25; 435/7.21; 435/325; 705/500

(57) ABSTRACT

The invention provides methods for identifying and isolating cells that express high levels of protein. A method is described for identifying a cell or cell line expressing a desired protein comprising determining in a cell or cell line that has been transfected with a vector comprising a nucleotide sequence encoding the desired protein the intracellular expression levels of elements of the vector and also determining expression of the desired protein on the surface of the cell or cell line, in certain cases in a single assay.

### RELATED APPLICATIONS

This application claims priority under 35 U.S.C. §119(e) to U.S. Provisional Patent Application No. 60/840,070, filed Aug. 23, 2006, and entitled “MULTI-VARIANT CELL INDICATION TECHNIQUE”, which is incorporated herein by reference.

### FIELD OF THE INVENTION

This invention pertains to the field of selection of cell lines that express proteins at enhanced levels.

### BACKGROUND

Proteins are important for a variety of processes ranging from therapeutic to industrial applications. Proteins for therapeutic applications include enzymes like alpha-galactosidase and diagnostic and therapeutic antibodies. Proteins can be produced in a variety of organisms including bacteria, yeast, fungi, insect cells, mammalian cells and in the milk of transgenic mammals. Proteins used in therapeutic applications often require posttranslational modifications, which necessitates production of these proteins in higher organisms.

The generation and establishment of mammalian cell lines for the production of proteins remains a challenge. Cell lines for protein production can be generated through the introduction of one or multiple copies of the gene coding for the desired protein into the genome of the cell. Once the gene is introduced into the cell, cells that integrate one or multiple copies of the gene into their genome are selected for. Selection is generally done by introducing the gene of interest in conjunction with a gene that protects the cell when that cell is challenged by a specific cytotoxic agent, like an antibiotic. While these techniques have utility for selecting cells that are capable of producing the desired protein product, they generally are not optimal for determining which populations of such cells are able to produce the proteins at the highest levels over the entire cell cycle of the cell. Accordingly, improvements in current methods for screening and selecting transfected cell populations that express desired proteins at high levels are desirable.

### SUMMARY OF THE INVENTION

Disclosed herein are inventive methods for the identification and/or selection of cells and cell lines that express relatively high levels of a desired protein.

One aspect of the invention is a method for identifying a cell or cell line expressing a desired protein comprising: determining in a cell or cell line that has been transfected with a vector comprising a nucleotide sequence encoding the desired protein the intracellular expression levels of elements of the vector; and determining expression of the desired protein on the surface of the cell or cell line; wherein both determining steps are performed in a single assay. In some embodiments the first determining step comprises determining the presence and relative abundance of the vector in a genome of the cell or cell line. In some embodiments both determining steps are performed essentially simultaneously. In some embodiments both determining steps are performed in the order recited.

In some embodiments the vector contains multiple copies of a nucleic acid sequence encoding the desired protein. In some embodiments the vector is integrated into the genome of the cell or cell line. In some embodiments multiple copies of the vector are integrated into the genome of the cell or cell line. In another embodiment the vector further comprises at least one copy of a nucleic acid sequence encoding dihydrofolate reductase (DHFR). In some embodiments the cell or cell line is deficient for dihydrofolate reductase (DHFR). The method may further comprise treating a cell or cell line with increasing doses of methotrexate or methotrexate analog.

In some embodiments the first determining step of the method comprises determining the expression level of dihydrofolate reductase (DHFR). In addition, this determining step can comprise determining the resistance of a cell or cell line to methotrexate or a methotrexate analog. In some embodiments determining the resistance of a cell or cell line to methotrexate comprises determining the amount of methotrexate or methotrexate analog that can be taken up into a live cell while retaining viability of the cell. In some embodiments the methotrexate or methotrexate analog comprises a fluorescent moiety.

In some embodiments the first determining step of the method comprises determining a gene copy number of the vector in the genome of the cell or cell line. This may be done by exposing the cell or cell line to a labeled probe able to specifically bind to the vector.

In some embodiments the second determining step of the method comprises determining expression of the desired protein on the surface of the cell or cell line by transient expression of the protein on the cell surface. Determining expression of the desired protein on the surface of the cell or cell line may be done, for example, by affinity matrix retention or by gel encapsulation. In some embodiments the method of determining the expression of a desired protein on the surface of the cell or cell line comprises contacting the cell or cell line with a first antibody having specific binding affinity to the desired protein. The first antibody may comprise a fluorescent moiety. In some embodiments the method may further comprise contacting the first antibody with a secondary antibody having specific binding affinity to the first antibody. The secondary antibody may comprise a fluorescent moiety.

In some embodiments of the method, the single assay for determining the intracellular expression levels of elements of the vector and the expression of the desired protein on the surface of the cells is a fluorescence based assay. This fluorescence based assay can be a flow cytometry assay.

In certain embodiments, the cell or cell line used for any of the assays can be a CHO cell or cell line.

In some embodiments the method further comprises isolating cells based on expression levels determined by the assay. The invention also embraces cell cultures and progeny comprising a population of cells enriched in the cells or cell line identified by any of the methods. In some embodiments the method further comprises isolating the desired protein expressed by any of the identified or isolated cells or cell lines.

Another aspect of the invention is a method for isolating a cell or cell line that has been treated with methotrexate or a methotrexate analog and that expresses a desired protein. The method comprises determining the intracellular expression levels of elements of a vector by determining the amount of methotrexate or methotrexate analog that can be taken up into a live cell while retaining viability of the cell; and determining the expression of the desired protein on the surface of the cell or cell line; wherein the cell or cell lines are isolated based on comparing the levels of intracellular expression levels of elements of a vector and the levels of the expression of the desired protein on the surface of the cell or cell line to selected levels. In some embodiments the levels of intracellular expression levels of elements of a vector and the levels of the expression of the desired protein on the surface of the cell or cell line are at least 10%, 50% or two times higher than the selected levels.

Another aspect of the invention is an algorithm comprising determining the selected levels of intracellular expression levels of elements of a vector and the levels of the expression of the desired protein on the surface of the cell or cell line necessary for isolating the cell or cell line of the preceding method, wherein the expression level of the desired protein of the isolated cell or cell line exceeds the expression level of the desired protein of an initial population of cells or cell lines from which the cell or cell line were isolated by at least about 50%. In addition the invention provides an article, comprising a machine-readable medium comprising a program, embodied in the medium, for causing a machine to determine the selected levels of intracellular expression levels of elements of a vector and the levels of the expression of the desired protein on the surface of the cell or cell line necessary for isolating the cell or cell line of the preceding method, wherein the expression level of the desired protein of the isolated cell or cell line exceeds the expression level of the desired protein of an initial population of cells or cell lines from which the cell or cell line were isolated by at least about 50%.

Another aspect of the invention is a business method comprising marketing or selling the cell or cell line identified in any of the preceding methods.

In one aspect the invention comprises kits for the selection and/or isolation of cells or cell lines expressing a desired protein. In some embodiments the kit comprises a vector, methotrexate or a methotrexate analog and instructions for use. In some embodiments of the kit, the vector comprises at least one copy of a nucleic acid sequence encoding dihydrofolate reductase (DHFR) and a copy of a nucleic acid encoding a desired protein. In some embodiments of the kit, the methotrexate or methotrexate analog is a fluorescent methotrexate or methotrexate analog.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as, optionally, additional items.

### DESCRIPTION OF THE FIGURES

FIG. 1 is an illustrative example of a dot-plot created for an individual cell pool analyzed; the y-axis represents FL1 values: the fluorescence intensities of FITC-labeled F-MTX; the x-axis represents FL2 values: fluorescence intensities of RPE-IgG.

### DETAILED DESCRIPTION OF THE INVENTION

Disclosed herein are inventive methods for the selection of cells or cell lines that express relatively high levels of a desired protein. In some embodiments the selected cells or cell line(s) has multiple copies of the gene coding for the desired protein integrated in its genome. In some embodiments the genes are integrated in a region of the host genome that is expressed. In some embodiments multiple copies of the gene coding for the desired protein are integrated in a region of the host genome that is expressed.

One non-limiting example of a method of introducing genes into a cell line that can be useful for practicing certain aspects of certain embodiments of the inventive methods comprises transfection of the cell line with a vector comprising the gene(s) encoding for a desired protein(s). In some embodiments the vector comprises more than one copy of the gene(s) coding for the desired protein(s), one or more selection markers, and additional genetic elements to express both the protein(s) of interest and the selection marker(s). Once a vector has been introduced into a cell or cell line, cells that have taken up the vector can be selected for. In some embodiments selection is done by challenging the cells with an agent which kills the cell unless the selection marker is being expressed. Non-limiting examples of selection markers than may be utilized in certain embodiments include genes that confer antibiotic resistance and genes coding for proteins involved in metabolism. Selection marker systems comprise combinations of the protective gene, like an antibiotic resistance marker, and the challenging agent, like an antibiotic. A variety of appropriate selection marker systems are known to those skilled in the art and may be used in the context of practicing certain embodiments of the invention.

One embodiment of a selection marker system useful for transfection in certain methods of the invention is the dihydrofolate reductase (DHFR) methotrexate (MTX) combination. See for instance Yoshikawa et al. (2000, Biotech. and Bioeng. 74: 435-442), which is hereby incorporated by reference. DHFR catalyses the reduction of dihydrofolate to tetrahydrofolate and is an essential protein for DNA synthesis. Effective inactivation of DHFR results in inhibition of DNA synthesis and cell death.

Methotrexate, abbreviated MTX, and formerly known as amethopterin, is an antimetabolite drug used in treatment of cancer and autoimmune diseases. It is a folate analogue that acts by inhibiting the metabolism of folic acid. Methotrexate competitively and reversibly inhibits dihydrofolate reductase (DHFR), an enzyme that is part of the folate synthesis metabolic pathway. The affinity of methotrexate for DHFR is about one thousand-fold that of folate for DHFR. Dihydrofolate reductase catalyses the conversion of dihydrofolate to the active tetrahydrofolate. Folic acid is needed for the de novo synthesis of the nucleoside thymidine, required for DNA synthesis. Methotrexate, therefore, inhibits the synthesis of DNA, RNA, thymidylates, and proteins. Methotrexate is cell cycle S-phase selective, and has a greater negative effect on rapidly dividing cells (such as malignant and myeloid cells), which are replicating their DNA, and thus inhibits the growth and proliferation of these cells.

In one embodiment of the selection marker system of the invention, cells that are deficient in DHFR are grown in media supplemented with nucleotides. In some embodiments the cell line deficient in DHFR is a CHO cell line. The DHFR deficient cells are subsequently transfected with a vector containing the gene for DHFR. In some embodiments the vector contains one or more copies of the gene for DHFR. In some embodiments the vector also contains one or more copies of a gene(s) coding for a desired protein(s). Upon transfection, the cells taking up and expressing the vector can be grown in media without nucleotides because the DNA synthesis pathway is restored. MTX can subsequently be used to select for transformants. Only cells that have taken up the DHFR gene will survive the challenge by MTX. In some embodiments transformation and selection results in one or more copies of the vector being integrated into the genome of the cell. In some embodiments the cell line is challenged by successive rounds of increasing concentrations of MTX. This invention, in certain embodiments, embraces use of MTX and/or any one or more of all structural analogs of MTX that specifically bind DHFR, as well as use of all agents with the same function as MTX, namely specific binding to DHFR.

In addition to being a tool for selection, MTX can also be used to determine DHFR expression through visualization. MTX binds to DHFR and the amount of MTX taken up in a live cell while retaining viability of the cell is a measure for the amount of expressed DHFR. If MTX is conjugated to a detectable label, such as a fluorescent label or moiety, the amount of MTX taken up in a cell can be visualized and correlates with the amount of expressed DHFR. In some embodiments determining the amount of MTX that is taken up by a cell, is the same as determining the resistance of the cell to MTX.

In some embodiments of the invention one or more known and commercially available cell lines deficient in DHFR are transfected with a vector comprising one or more genes encoding for DHFR and a desired protein. Transformants are subsequently selected for by challenging the cells with MTX. In some embodiments the level of DHFR expression is determined by exposing the cells to MTX conjugated to a fluorescent moiety and determining binding of the MTX to DHFR. The expression of DHFR will correlate with the expression of the desired protein, since both were introduced on the same vector. Thus, this assay is an indirect determinant of the production of the desired protein. It determines the protein expression capability of the cell, but it does not determine the amount of the desired protein actually expressed by the cell. For instance, it is possible that the desired protein was recombined out sometime during the transformation and integration process or was not properly folded or secreted, leading to degradation and therefore the production of desired protein no longer correlates with the amount of expressed DHFR.

In some embodiments determining the intracellular levels of expression of a desired protein is inferred by simply determining the presence and relative abundance of the vector in the genome of a cell or cell line. In some embodiments the copy number of the vector in the genome of the cell or cell line is determined. In some embodiments determination is performed by binding a labeled probe that specifically binds to the gene encoding the desired protein, to the genome of the cell or cell line. Such techniques are standard and well known to those skilled in the art.

The invention involves, in certain embodiments, combining assays for determining the presence and relative abundance of the vector in the genome of a cell or cell line and/or the expression of a marker, as described above, with an additional assay(s)/determinations that comprise identifying and/or selecting productive cells by their ability to produce and secrete the desired protein. Expression of the desired protein can be determined, for example, by a variety of techniques known in the art, such as by using various known techniques for measuring the level of the desired protein on the cell surface. In some embodiments expression on the surface is determined by transient expression of the protein, or a portion thereof, on the cell surface. In other embodiments expression of the desired protein on the cell surface is determined by affinity matrix retention techniques. In yet other embodiments, expression of the desired protein on the cell surface is determined by gel encapsulation techniques. Each of these techniques is described further below.

In some embodiments expression of proteins is determined by transient expression of the protein on the cell surface. This method is described in Brezinsky et al. (2003 J. Immunol. Meth. 277:141-155), which is incorporated herein by reference. In this technique, transfected cells are exposed to low temperature conditions to “freeze” or slow the secretion process and transit of the protein through the membrane. The method comprises visualizing the proteins at the time of secretion. Proteins are synthesized on ribosomes, transferred to the Golgi apparatus and then to the plasma membrane where the secreted protein is released from secretory vessels through the plasma membrane into the surrounding milieu. Association of the protein with the cell surface is downstream of the rate-limiting step and the protein on the cell surface correlates with the amount of protein being produced by the cell. The proteins on the cell surface are visualized through low temperature staining. Cells to be analyzed are harvested and then maintained at 0-4° C. The cells are subsequently stained with an antibody that specifically binds to the secreted desired protein. For example, if the secreted desired protein contains a human IgG domain, the protein can be visualized by an anti-human IgG antibody. In some embodiments the antibody can be visualized by conjugating the antibody to a fluorescent or other detectable marker or moiety. In other embodiments the antibody can be visualized by binding the primary antibody to the desired protein with a secondary antibody specific to the primary antibody, the secondary antibody being conjugated to a fluorescent or other detectable marker or moiety. For embodiments where the antibody is labeled with a fluorescent marker or moiety, the fluorescent marker or moiety can subsequently be analyzed under a fluorescent microscope or any other device that can detect fluorescence, whereby the intensity of fluorescence correlates with, and is a method of determining, expression of the desired protein. Fluorescent markers also can facilitate identifying and isolating fluorescent cells with Fluorescence Activated Cell Sorting (FACS), as discussed in detail below.

In some embodiments expression of proteins is determined by affinity matrix retention techniques. This method is described in Borth et al. (2000 Biotechnology and Bioengineering 71:266-273), which is incorporated herein by reference. Affinity matrix retention is also based on visualizing the protein when it is being secreted. The secreted desired protein is captured by covering the cells with a surface affinity matrix, which captures the secreted protein product with antibodies that specifically bind the desired protein and that are linked to cell a surface protein via biotin-avidin bridges. The affinity matrix may be applied by incubating cells in a solution containing sulfo-NHS-biotin and subsequently incubating the cells in a solution containing avidin. The last step comprises incubating the cells with a solution of biotin-antibody conjugate, wherein the antibody can specifically bind the protein of interest. As described in the low temperature transit assay (above) the antibody can be conjugated to a fluorescent marker or moiety and thus visualized directly, or the antibody can be visualized by binding it to a secondary antibody that is conjugated to a fluorescent marker or moiety. The intensity of fluorescence correlates with, and is a method of determining, protein expression. Fluorescent markers also can facilitate identifying and isolating fluorescent cells with Fluorescence Activated Cell Sorting (FACS).

In some embodiments expression of proteins is determined by gel encapsulation. This method is described in U.S. Pat. No. 6,806,058 (Jesperson) which is incorporated herein by reference. One embodiment of the method entails encapsulating a cell in a microdrop wherein the microdrop comprises matrix component molecules, including biotin molecules and capture molecules. One embodiment of capture molecules are antibodies that specifically bind proteins secreted by the cell. In some embodiments both the matrix molecules and the capture molecules are linked to biotin molecules, with streptavidin linking both biotin molecules, resulting in an encapsulating gel matrix. When the desired protein is secreted from the cell it binds to the capture molecules and is thereby retained within the microdrop. The secreted molecule can be detected by conjugating the capture molecule (the antibody) to a fluorescent marker or moiety, or by binding the capture molecule to a secondary antibody that is conjugated to a fluorescent marker or moiety. The intensity of fluorescence correlates with, and is a method of, determining protein expression. Fluorescent markers also can facilitate identifying and isolating fluorescent cells with Fluorescence Activated Cell Sorting (FACS).

The embodiments for visualizing secreted proteins described above, provide a method to determine the protein expression activity and actual protein production of the cell. However, when employed alone, assays for determining the amount of secreted protein, since it is cell cycle dependent, provide only a one-time assessment of protein production potential of a cell or cell line at a particular instant in the cell cycle. Determination of the amount of protein on the cell surface alone does not provide a long term picture of protein expression capacity of a specific cell or cell line. As previously discussed, the method of determining the intracellular expression levels of elements of a transfected vector, of which the DHFR/MTX method of the invention is one embodiment, when employed alone, provides a long term picture of the innate protein production capacity of a cell but does not always correlate with actual production and secretion of the desired protein.

Thus, as previously mentioned, certain embodiments of the invention provide methods for identifying and/or isolating a cell population or cell line expressing, producing and secreting a desired protein by combining the determination of intracellular expression levels of the desired protein with the determination of cell surface expression levels of the desired protein in one single assay or determination. In some embodiments this single assay is performed by visualizing the amount of the intracellular expression and the surface expression level of a desired protein through the use of fluorescence labeling. In some embodiments the single assay is a fluorescence assay. In some embodiments the fluorescence assay is a flow cytometry or FACS assay.

Flow cytometry allows for the analysis of large numbers of cells in a short period of time. Newly developed flow cytometers can analyze and sort up to 100,000 cells per second. In a typical flow cytometer, individual particles or cells pass through an illumination zone and appropriate detectors, gated electronically, measure the magnitude of a pulse representing the extent of light scattered. The magnitude of these pulses are sorted electronically into “bins” or “channels”, permitting the display of histograms of the number of cells possessing a certain quantitative property versus the channel number. The data accruing from cytometric measurements are analyzed rapidly allowing for electronic cell-sorting procedures. This can be used to sort cells with desired properties into separate “buckets”, a procedure usually known as Fluorescence-Activated Cell Sorting (FACS).

Fluorescence-activated cell sorting has been primarily used in studies of human and animal cell lines and in the control of cell culture processes. Fluorophore labeling of cells and measurement of the fluorescence can give quantitative data about specific proteins, target molecules or subcellular components and their distribution in the cell population. Flow cytometry can quantitate virtually any cell-associated property or cell organelle for which there is a fluorescent probe (or natural fluorescence).

In certain embodiments, the use of FACS allows for the visualization of more than one fluorescent marker at the same time and the ability to enrich cells characterized by the presence of high levels of multiple markers associated with the cells. By, for example, exposing transfected cells exposed to a fluorescently labeled MTX, for cells transfected with a vector comprising a marker gene coding for DHFR, in combination with a fluorescently labeled antibody to the desired protein, a FACS assay can determine both the intracellular expression level and the expression of the protein on the surface at the same time. Such a FACS assay can thereby identify cells according to their ability to produce protein at the highest levels. In some embodiments the FACS assay is performed by identifying cells based on both intracellular and surface expression protein expression parameters in a single experiment, test, step or assay, such as a single screening/selection run on a FACS machine. In certain embodiments, the determinations for both intracellular and surface expression protein expression parameters may be performed essentially simultaneously, while in other embodiments, they may be performed sequentially in any desired order. In certain embodiments, both intracellular and surface expression protein expression parameters are determined without a substantial delay between determination of intracellular expression/gene copy parameters and determination of surface expression parameters, for example of less than one day, less than 1 hour, less than 30 min., less than 15 min, less than 10 min., less than 5 min., less than 1 min., less than 30 sec., less than 10 sec., less than 5 sec., less than 1 sec., or essentially no delay (i.e., essentially simultaneously).

FACS allows for the isolation of cells that have certain fluorescence levels. Cells that have been identified based on the determination of the two expression parameters discussed above can thus be isolated. This isolation process can result in a population of cells enriched for cells that express a desired protein at levels substantially higher than the average level of production of the initial transfected population. The invention embraces both the cells isolated by such an isolation procedure and the progeny of these cells. In addition, the invention embraces isolating a desired protein produced by the identified and/or isolated cells.

In some embodiments the determination of appropriate protein expression levels for selecting cells is made by comparing expression levels to a selected threshold level. The selected level can be set by comparing the fluorescent signal of a cell expressing the desired protein, to a fluorescent signal of cells that do not express the desired protein or express the protein at a particular level. In some embodiments cell lines are isolated based on comparing measured levels of fluorescence to selected levels of fluorescence. In some embodiments cells that have a fluorescence of at least 10% higher than selected levels are isolated. In some embodiments cells that have a fluorescence of at least 20% higher than selected levels are isolated. In some embodiments cells that have a fluorescence of at least 30% higher than selected levels are isolated. In some embodiments cells that have a fluorescence of at least 40% higher than selected levels are isolated. In some embodiments cells that have a fluorescence of at least 50% higher than selected levels are isolated. In some embodiments cells that have a fluorescence of at least two times higher than selected levels are isolated. In some embodiments cells that have a fluorescence of at least five times higher than selected levels are isolated. In some embodiments cells that have a fluorescence of at least ten times higher than selected levels are isolated. In certain embodiments, the selected level will be a level that is higher by some factor than an average level determined for the initial population of transfected cells, such as 10% higher, 20%, 30%, 40%, 50%, 100%, 200%, 500%, 1000% or more higher than the level of an average producer of the initial population.

In some embodiments an algorithm may be used to determine the selected levels of expression necessary for isolating a cell or cell line expressing the protein of interest at a particular desired level. In some embodiments an algorithm may be used to assess if the expression level of the desired protein of the isolated cell or cell line exceeds the expression level of the desired protein of an initial population of cells or cell lines from which the cell or cell line were isolated by a selected amount. In some embodiments the algorithm will be based on comparing averages and mean values of fluorescence in cell populations. In some embodiments the algorithm will be based on the number of cell populations, the number of cells, and the range of signal intensity. It should be appreciated that invention also embraces computer implementation of the algorithm. In some embodiments the invention comprises a machine-readable medium comprising a program, embodied in the medium, for causing a machine to determine selected levels necessary for isolating a cell or cell line expressing the desired protein.

Below is described certain techniques for performing certain procedures that may be useful for practicing certain embodiments of the invention.

### Vectors, Gene Expression and Transfection

Certain aspects of the invention may involve the use of sequences that encode a protein, peptide or fragment or variant thereof, in expression vectors, as well their use to transfect host cells and cell lines, be these prokaryotic (e.g., E. coli), or eukaryotic (e.g., CHO cells, COS cells, yeast expression systems and recombinant baculovirus expression in insect cells). Especially useful are mammalian cells such as human, mouse, hamster, pig, goat, primate, etc. The cells may be of a wide variety of tissue types, and they may be primary cells or cell lines. The expression vectors may include the pertinent sequence, i.e., those encoding desired peptide sequences, operably linked to a promoter.

In certain embodiments, expression vectors comprising any isolated nucleic acid molecules for coding a desired protein, preferably operably linked to a promoter, are provided. Host cells transformed or transfected with such expression vectors may also be provided. Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA (or RNA) encoding a desired protein. The heterologous DNA (or RNA) is placed under operable control of transcriptional elements to permit the expression of the heterologous DNA in the host cell.

As used herein, a “vector” may be any of a number of nucleic acid molecules into which a desired sequence may be inserted by restriction and ligation for transport between different genetic environments or for expression in a host cell. Vectors are typically composed of DNA although RNA vectors are also available. Vectors include, but are not limited to, plasmids, phagemids and virus genomes. A cloning vector is one which is able to replicate in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host cell or just a single time per host before the host reproduces. In the case of phage, replication may occur actively during a lytic phase or passively during a lysogenic phase.

An “expression vector” is one into which a desired DNA sequence may be inserted by restriction and ligation such that it is operably joined to regulatory sequences and may be expressed as an RNA transcript. Vectors may further contain one or more marker sequences, as discussed above, suitable for use in the identification of cells that have or have not been transformed or transfected with the vector. Markers include, for example, genes encoding proteins that increase or decrease either resistance or sensitivity to antibiotics or other compounds, genes that encode enzymes whose activities are detectable by standard assays known in the art (e.g., beta-galactosidase or alkaline phosphatase), and genes that visibly affect the phenotype of transformed or transfected cells, hosts, colonies or plaques (e.g., green fluorescent protein). Any of these markers could potentially be used a basis for the above described methods of determining gene copy and/or intracellular expression levels of the transfected vector. Certain vectors are capable of autonomous replication and expression of the structural gene products present in the DNA segments to which they are operably joined.

As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript might be translated into the desired protein or polypeptide.

The precise nature of the regulatory sequences needed for gene expression may vary between species or cell types, but generally include, when necessary, 5′ non-transcribed and 5′ non-translated sequences involved with the initiation of transcription and translation respectively, such as a TATA box, capping sequence, CAAT sequence, and the like. Especially, such 5′ non-transcribed regulatory sequences will include a promoter region that includes a promoter sequence for transcriptional control of the operably joined gene. Regulatory sequences may also include enhancer sequences or upstream activator sequences as desired. The vectors may optionally include 5′ leader or signal sequences. The choice and design of an appropriate vector is within the ability and discretion of one of ordinary skill in the art.

In this respect, for example, the person skilled in the art will readily appreciate that the polynucleotides encoding at least the variable domain of the light and/or heavy chain may encode the variable domains of both immunoglobulin chains or only one. Likewise, polynucleotides may be under the control of the same promoter or may be separately controlled for expression. Possible regulatory elements permitting expression in prokaryotic host cells comprise, e.g., the PL, lac, trp or tac promoter in E. coli, and examples of regulatory elements permitting expression in eukaryotic host cells are the AOX1 or GAL1 promoter in yeast or the CMV-promoter, SV40-promoter, RSV-promoter (Rous sarcoma virus), CMV-enhancer, SV40-enhancer or a globin intron in mammalian and other animal cells.

Beside elements which are responsible for the initiation of transcription such regulatory elements may also comprise transcription termination signals, such as the SV40-poly-A site or the tk-poly-A site, downstream of the polynucleotide. Furthermore, depending on the expression system used, leader sequences capable of directing the polypeptide to a cellular compartment, or secreting it into the medium may be added to the coding sequence of the polynucleotides and are well known in the art. The leader sequence(s) is (are) assembled in appropriate phase with translation, initiation and termination sequences, and in certain embodiments, a leader sequence capable of directing the polypeptide to a cellular compartment or directing secretion of translated protein, or a portion thereof, into the periplasmic space or extracellular medium. In this context, suitable expression vectors are known in the art such as Okayama-Berg cDNA expression vector pcDV1 (GE Healthcare (Piscataway, N.J.), pCDM8, pRc/CMV, pcDNA1, pcDNA3 (Invitrogen, Carlsbad, Calif.), or pSPORT1 (Invitrogen, Carlsbad, Calif.).

The present invention furthermore makes use of, in certain embodiments, host cells transformed with a polynucleotide or vector encoding a desired protein. Such host cell may be a prokaryotic or eukaryotic cells. The polynucleotide or vector which is present in the host cell may either be integrated into the genome of the host cell or it may be maintained extrachromosomally. The host cell can be any prokaryotic or eukaryotic cell, such as a bacterial, insect, fungal, plant, animal or human cell. Certain fungal cells are, for example, those of the genus Saccharomyces, in particular those of the species S. cerevisiae. The term “prokaryotic” is meant to include all bacteria which can be transformed or transfected with DNA or RNA molecules for the expression of a peptide sequence of the invention. Prokaryotic hosts may include gram negative as well as gram positive bacteria such as, for example, E. coli, S. typhimurium, Serratia marcescens and Bacillus subtilis. The term “eukaryotic” is meant to include yeast, higher plant, insect and in certain embodiments mammalian cells, for example NSO and CHO cells. Depending upon the host employed in a recombinant production procedure, the peptide sequences encoded by the polynucleotides of the vector may be glycosylated or may be non-glycosylated. A polynucleotide or vector can be used to transform or transfect the host using any of the techniques commonly known to those of ordinary skill in the art. Furthermore, methods for preparing fused, operably linked genes and expressing them in, e.g., mammalian cells and bacteria are well-known in the art (Sambrook, Molecular Cloning: A Laboratory Manual, Cold Spring Harbor Laboratory, Cold Spring Harbor, N.Y., 1989). The genetic constructs and methods described therein, or straightforward modifications thereof, can be utilized for expression of the proteins or polypeptides in eukaryotic or prokaryotic hosts. In certain embodiments, expression vectors containing promoter sequences which facilitate the efficient transcription of the inserted polynucleotide are used in connection with the host. The expression vector typically contains an origin of replication, a promoter, and a terminator, as well as specific genes which are capable of providing phenotypic selection of the transformed cells. Suitable source cells for the DNA sequences and host cells for polypeptide expression can be obtained from a number of sources, such as the American Type Culture Collection (see, www.atcc.org).

In some embodiments, a virus vector for delivering a nucleic acid molecule encoding a protein or peptide sequence is selected from the group consisting of adenoviruses, adeno-associated viruses, poxviruses including vaccinia viruses and attenuated poxviruses, Semliki Forest virus, Venezuelan equine encephalitis virus, retroviruses, Sindbis virus, and Ty virus-like particle. Examples of viruses and virus-like particles which have been used to deliver exogenous nucleic acids include: replication-defective adenoviruses (e.g., Xiang et al., Virology 219:220-227, 1996; Eloit et al., J. Virol. 7:5375-5381, 1997; Chengalvala et al., Vaccine 15:335-339, 1997), a modified retrovirus (Townsend et al., J. Virol. 71:3365-3374, 1997), a nonreplicating retrovirus (Irwin et al., J. Virol. 68:5036-5044, 1994), a replication defective Semliki Forest virus (Zhao et al., Proc. Natl. Acad. Sci. USA 92:3009-3013, 1995), canarypox virus and highly attenuated vaccinia virus derivative (Paoletti, Proc. Natl. Acad. Sci. USA 93:11349-11353, 1996), non-replicative vaccinia virus (Moss, Proc. Natl. Acad. Sci. USA 93:11341-11348, 1996), replicative vaccinia virus (Moss, Dev. Biol. Stand. 82:55-63, 1994), Venezuelan equine encephalitis virus (Davis et al., J. Virol. 70:3781-3787, 1996), Sindbis virus (Pugachev et al., Virology 212:587-594, 1995), and Ty virus-like particle (Allsopp et al., Eur. J. Immunol 26:1951-1959, 1996). In certain embodiments, the virus vector is an adenovirus.

Another virus, which can potentially be used for certain applications, is the adeno-associated virus, a double-stranded DNA virus. The adeno-associated virus is capable of infecting a wide range of cell types and species and can be engineered to be replication-deficient. It further has advantages, such as heat and lipid solvent stability, high transduction frequencies in cells of diverse lineages, including hematopoietic cells, and lack of superinfection inhibition thus allowing multiple series of transductions. The adeno-associated virus can integrate into human cellular DNA in a site-specific manner, thereby minimizing the possibility of insertional mutagenesis and variability of inserted gene expression. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA. Adenoviruses and retroviruses have been approved for human gene therapy trials. In general, the retroviruses are replication-deficient (i.e., capable of directing synthesis of the desired proteins, but incapable of manufacturing an infectious particle). Such genetically altered retroviral expression vectors can have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, M., Gene Transfer and Expression, A Laboratory Manual, W.H. Freeman Co., New York (1990) and Murry, E. J. Ed. “Methods in Molecular Biology,” vol. 7, Humana Press, Inc., Clifton, N.J. (1991).

In certain embodiments, the foregoing nucleic acid delivery vectors: (1) contain exogenous genetic material that can be transcribed and translated in a mammalian cell and that can produce a desired protein or peptide sequence, and (2) contain on a surface a ligand that selectively binds to a receptor on the surface of a target cell, such as a mammalian cell, and thereby gains entry to the target cell.

Various techniques may be employed for introducing nucleic acid molecules of the invention into cells, depending on whether the nucleic acid molecules are introduced in vitro or in vivo in a host. Such techniques include transfection of nucleic acid molecule-calcium phosphate precipitates, electroporation, transfection of nucleic acid molecules associated with DEAE, transfection or infection with the foregoing viruses including the nucleic acid molecule of interest, liposome-mediated transfection, and the like.

For certain uses, it is preferred to target the nucleic acid molecule to particular cells. In such instances, a vehicle used for delivering a nucleic acid molecule of the invention into a cell (e.g., a retrovirus, or other virus; a liposome) can have a targeting molecule attached thereto. For example, a molecule such as an antibody specific for a surface membrane protein on the target cell or a ligand for a receptor on the target cell can be bound to or incorporated within the nucleic acid molecule delivery vehicle. Useful in this context are monoclonal antibodies. Where liposomes are employed to deliver the nucleic acid molecules, proteins that bind to a surface membrane protein associated with endocytosis may be incorporated into the liposome formulation for targeting and/or to facilitate uptake. Such proteins include capsid proteins or fragments thereof specific for a particular cell type, antibodies for proteins which undergo internalization in cycling, proteins that target intracellular localization and enhance intracellular half life, and the like. Polymeric delivery systems also have been used successfully to deliver nucleic acid molecules into cells, as is known by those skilled in the art. Such systems even permit oral delivery of nucleic acid molecules.

In addition to delivery through the use of vectors, nucleic acids of the invention may be delivered to cells without vectors, e.g., as “naked” nucleic acid delivery using methods known to those of skill in the art.

In certain embodiments of the invention, a transgenic non-human animal comprising an expression vector may be used. As used herein, “transgenic non-human animals” includes non-human animals having one or more exogenous nucleic acid molecules incorporated in germ line cells and/or somatic cells. Thus the transgenic animal include animals having episomal or chromosomally incorporated expression vectors, etc. In general, such expression vectors can use a variety of promoters which confer the desired gene expression pattern (e.g., temporal or spatial). Conditional promoters also can be operably linked to nucleic acid molecules of the invention to increase or decrease expression of the encoded polypeptide molecule in a regulated or conditional manner. Trans-acting negative or positive regulators of polypeptide activity or expression also can be operably linked to a conditional promoter as described above. Such trans-acting regulators include antisense nucleic acid molecules, nucleic acid molecules that encode dominant negative molecules, transcription factors, ribozyme molecules specific for nucleic acid molecules, and the like. The transgenic non-human animals may be useful in experiments directed toward testing biochemical or physiological effects of diagnostics or therapeutics. Other uses will be apparent to one of ordinary skill in the art.

The invention also may utilize so-called expression kits, which allow the artisan to prepare a desired expression vector or vectors. Such expression kits may include at least one nucleotide sequence encoding a desired protein or peptide sequence. Other components may be added, as desired, as long as the above-mentioned sequences are included.

Suitable methods of detecting molecules include, without limitation, the detection of fluorescent, chemiluminescent or radioactive molecules or molecules bound to a fluorescent, chemiluminescent or radioactive marker, measurement of molecular concentrations in solution.

A fluorescent label, a fluorescent moiety or fluorescent marker or fluorophore is a substance which is capable of exhibiting fluorescence within a detectable range. Fluorophores include, but are not limited to, fluorescein, isothiocyanate, fluorescein amine, eosin, rhodamine, dansyl, umbelliferone, 5-carboxyfluorescein (FAM), 2′7′-dimethoxy-4′5′-dichloro-6-carboxyfluorescein (JOE), rhodamine, 6 carboxyrhodamine (R6G), N,N,N′,N′-tetramethyl-6-carboxyrhodamine (TAMRA), 6-carboxy-X-rhodamine (ROX), 4-(4′-dimethylaminophenylazo) benzoic acid (DABCYL), 5-(2′-aminoethyl) aminonaphthalene-1-sulfonic acid (EDANS), 4-acetamido-4′-isothiocyanatostilbene-2,2′disulfonic acid, acridine, acridine isothiocyanate, r-amino-N->3-vinylsulfonyl)phenyl!naphthalimide-3,5, disulfonate (Lucifer Yellow VS), N-(4-anilino-1-naphthyl)maleimide, anthranilamide, Brilliant Yellow, coumarin, 7-amino-4-methylcoumarin, 7-amino-4-trifluoromethylcouluarin (Coumaran 151), cyanosine, 4′,6-diaminidino-2-phenylindole (DAPI), 5′,5″-diaminidino-2-phenylindole (DAPI), 5′,5″-dibromopyrogallol-sulfonephthalein (Bromopyrogallol Red), 7-diethylamino-3-(4′-isothiocyanatophenyl)-4-methylcoumarin diethylenetriamine pentaacetate, 4,4′-diisothiocyanatodihydro-stilbene-2,2′-disulfonic acid, 4,4′-diisothiocyanatostilbene-2,2′-disulfonic acid, 4-dimethylaminophenylazophenyl-4′-isothiocyanate (DABITC), eosin isothiocyanate, erythrosin B, erythrosin isothiocyanate, ethidium, 5-(4,6-dichlorotriazin-2-yl) aminofluorescein (DTAF), QFITC (XRITC), fluorescamine, IR144, IR1446, Malachite Green isothiocyanate, 4-methylumbelliferone, ortho cresolphthalein, nitrotyrosine, pararosaniline, Phenol Red, B-phycoerythrin, o-phthaldialdehyde, pyrene, pyrene butyrate, succinimidyl 1-pyrene butyrate, Reactive Red 4 (Cibacron® Brilliant Red 3B-A), lissamine rhodamine B sulfonyl chloride, rhodamine B, rhodamine 123, rhodamine X isothiocyanate, sulforhodamine B, sulforhodamine 101, sulfonyl chloride derivative of sulforhodamine 101, (Texas Red), tetramethyl rhodamine, tetramethyl rhodamine isothiocyanate (TRITC), riboflavin, rosolic acid, and terbium chelate derivatives.

As used herein, “copy number” is given its ordinary meaning as used in the art, i.e., the number of times a certain nucleic acid sequence appears within a genome. The copy numbers of regions within a genome are altered by events that amplify or delete sequences or subsequences within the genome. Variations in copy number detectable by the methods of the invention may arise in different ways. For example, copy number may vary as a result of amplification or deletion of a chromosomal region, e.g., as commonly occurs in cancer. Other variations are germ line genomic differences that are inherited through ancestors. Still other de novo variations arise spontaneously during mitosis or meiosis. Techniques for determining the copy number of the target nucleic acid were discussed in more detail above and are well known in the art.

As used herein, a cell is “viable” or in a “viable state” if the cell is able to perform normal or active physiological functions or activities for that type of cell. Examples of normal physiological functions that can be readily identified by those of ordinary skill in the art and include, but are not limited to, metabolism of certain substrates, synthesis of certain proteins, migration, mitosis, differentiation, etc. Those of ordinary skill in the art will be able to identify specific test(s) and/or assay(s) for determining the viability of any given cell or cell type.

A “kit,” as used herein, defines a package including any one or a combination of compositions, materials, etc. and instructions, but can also include compositions, materials, etc. and instructions of any form that are provided in connection with the compositions, materials, etc. in a manner such that a professional will clearly recognize that the instructions are to be associated with the compositions, materials, etc. The kits described herein may also contain, in some cases, one or more containers, which can contain compositions, materials, etc. The kits also may contain instructions for mixing, diluting, and/or administrating the compositions, materials, etc. The kits also can include other containers with one or more solvents, surfactants, preservative and/or diluents as well as containers for mixing, diluting or administering compositions, materials, etc. to cells or cell lines. In some embodiments the kit comprises a vector, a methotrexate or methotrexate analog and instructions for use. In some embodiments of the kit the vector comprises at least one copy of a nucleic acid sequence encoding dihydrofolate reductase (DHFR) and a copy of a nucleic acid encoding a desired protein. In some embodiments of the kit the methotrexate or methotrexate analog is a fluorescent methotrexate or methotrexate analog.

The compositions of the kit may be provided as any suitable form, for example, as liquid solutions or as dried powders. When the composition provided is a dry powder, the composition may be reconstituted by the addition of a suitable solvent, which may also be provided. In embodiments where liquid forms of the composition are used, the liquid form may be concentrated or ready to use. The solvent will depend on the formulation of the composition and the mode of use or administration. Suitable solvents are well known and are available in the literature.

The kit, in one set of embodiments, may comprise a carrier that is compartmentalized to receive in close confinement one or more container means such as vials, tubes, and the like, each of the compartments comprising one of the separate elements to be used in a method. Additionally, the kit may include containers for other components of the compositions, for example, buffers useful in the methods described herein.

### Business Applications:

Aspects of the invention may be useful to identify and isolate cells or cell lines that produce a desired protein. Accordingly, aspects of the invention relate to marketing methods, compositions, kits, devices, and systems related to the identified and/or isolated cells or cell lines described herein.

Aspects of the invention may be useful for reducing the time and/or cost of identifying and isolating cells or cell lines that produce a desired protein. Accordingly, aspects of the invention relate to business methods that involve collaboratively (e.g., with a partner) or independently marketing one or more cells or cell lines that produce a desired protein identified and/or isolated as described herein. For example, certain embodiments of the invention may involve marketing a procedure and/or associated devices or systems for identifying and/or isolating cells or cell lines that produce a desired protein.

Marketing may involve providing information and/or samples relating to methods, kits, compositions, devices, and/or systems described herein. Potential customers or partners may be, for example, companies in the pharmaceutical, biotechnology and agricultural industries, as well as academic centers and government research organizations or institutes. Business applications also may involve generating revenue through sales and/or licenses of methods, kits, compositions, devices, and/or systems of the invention.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

### EXAMPLES

Fluorescein-MTX (F-MTX; Invitrogen, Carlsbad, Calif.) can be used to identify high expressing cells on the basis of high fluorescence and DHFR gene copy numbers: both indirect markers of productivity. Presented here is a combination of the MTX method to identify and select cells coupled with a direct marker of productivity: fluorescent antibody to a cell surface molecule. A method is presented that allows for the evaluation of single clones on the basis of two distinct characteristics. Using such an approach results in the selectively capture of individual cells simultaneously possessing high DHFR gene copies (using F-MTX) and expressing transiently or constitutively high levels of surface protein (using RPE-conjugated mAb).

### Cell Sorting

### Staining of Cells

Cultures of CHO cells transfected by electroporation or lipofection with a vector comprising DHFR and a fusion-protein containing a human IgG fragment were maintained under MTX selective pressure and were stained prior to analytical analysis and/or sorting by flow cytometry. Fluorescein-MTX (F-MTX) was used to qualitatively identify cells in a transfected population that contained the highest expression of DHFR. F-MTX is absorbed by cells by the same mechanism as the unlabeled molecule and thus becomes an internal stain. Because F-MTX binds DHFR more weakly than non-labeled methotrexate, amplified cultures were first cleansed of their residual levels of internal MTX before being stained with F-MTX. Therefore, prior to staining amplified cells were subjected to a 3 day long “wash out”. Cells were collected, centrifuged, and the cell pellets re-suspended in MTX-free Excell-302 Selection Medium (JRH Biosciences, Lenexa, Kans.). The cultures were maintained in MTX-free Selection Medium for an additional two days and washed on a daily basis. On the third day after the final wash the cells (at a density of 1×10⁶ cells/mL) were placed in F-MTX containing MTX-free Selection Medium and stained overnight. The next day the samples were washed again and then analyzed or sorted immediately. Each sort required approximately 10-30 million cells per sample, while analytical flow cytometry required a minimum of 1 million cells per sample.

Cells were stained for specific surface molecule using R-Phycoerythrin-conjugated AffiniPure F(ab)₂ Fragment Goat anti-Human IgG (IgG-RPE) (Jackson ImmunoResearch Laboratories Inc, West Grove, Pa.). A solution of cold 2% gamma irradiated triple filtered dialyzed Fetal Bovine Serum (dFBS) in 1×PBS was used in washing steps and as an antibody diluent. Washed samples were stained in the dark for 15 minutes with a 1:100 dilution of cold antibody. The dFBS was obtained from Hyclone (Logan, Utah). R-Phycoerythrin-conjugated ChromPure Goat IgG F(ab)₂ fragment was used as an istotype control.

### Sorting of Cells

Analytical flow cytometry and bulk sorting were performed on a MoFlo high speed flow cytometer (DakoCytomation, Glostrup, Denmark) equipped with a single 488 nm Argon laser and D488/10, D530/40 and 570/40 band pass filters for the photo multiplier tubes. The fluorescence in signal one (FL1) was detected using a 555 DC long pass filter, and FL2 was detected using a 610 DC long pass filter. A dead-cell and debris exclusion gate was set using side scatter vs. forward-scatter, a doublet-exclusion gate was set up using pulse-width, and a boolean gate was set on various regions of FL1 and/or FL2 fluorescence intensity. Cloning was achieved by manual seeding of serial dilutions. The MoFlo was operated under a softwall HEPA filtered portable clean room enclosure.

Preliminary analytical flow cytometry studies were performed on 8 different cell populations using F-MTX and cell surface antibody. The entire panel of cell populations was tested (50 nM, 10 nM, 200 nM, 300 nM, 400 nM and 500 nM treatment with MTX) along with the bulk sorted pooled cell population (bulk sorts of populations grown in 100-500 nM MTX) and the bulk sort of 400 nM MTX population used in Limit Dilution Cloning-2 (LD2).

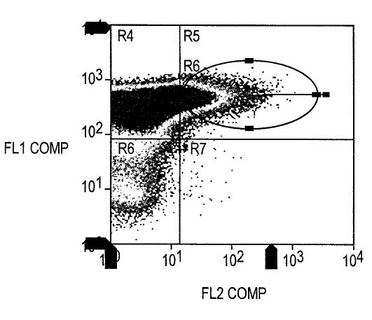

As demonstrated in the FIG. 1, high F-MTX fluorescing cells stained with the RPE conjugated antibody revealed two discrete populations contained within a band of IgG staining intensities; those positive for staining with RPE-IgG and those negative for staining with RPE-IgG. The observation suggests that a significant percentage of high fluorescing cells were not co-expressing surface antibodies. Table 1 displays the peak mean fluorescence intensity (PMF) of the IgG F-MTX population along with the percent of double positive (IgG/F-MTX) present for each of the samples tested. The populations that showed the greatest proportion of double-positive cells were in the 50 nM, 100 nM and 200 nM MTX pools: with the greatest peak mean fluorescence seen in the 200 nM pool.

TABLE 1

| Region | Count | % | % Events | Mean |
|---|---|---|---|---|
| Total | 1362510 | 100.00 | 66.05 | 7.25, 366.51 |
| R4 | 1144239 | 83.98 | 55.47 | 4.61, 372.47 |
| R5 | 152474 | 11.19 | 7.39 | 29.10, 466.16 |
| R6 | 65558 | 4.81 | 3.18 | 2.32, 31.90 |
| R7 | 239 | 0.02 | 0.01 | 32.84, 41.30 |
| R8 | 136691 | 10.03 | 6.63 | 30.64, 479.51 |

The results shown in Table 1 supported the decision to use the 200 nM MTX cell population for the subsequent staining and sort experiments. Three separately stained groups were generated: F-MTX (internal stain), RPE-conjugated anti-IgG (surface stain), and a combination of both. The 200 nM MTX culture was washed 3 days and divided into two groups (unstained and F-MTX stained) and incubated overnight. Fifty million cells were then stained with F-MTX in Selection Medium; while 25 million cells remained unstained. The next day the F-MTX internally stained group was washed and subdivided: one stained for surface IgG and the other not surfaced stained. Staining with goat anti-human IgG-conjugated RPE molecule involved exposing cells to animal derived materials in the form of the goat derived antibody and the Bovine Serum Albumin (BSA) added to the diluent.

Prior to conducting the bulk sorts a new MoFlo flow path was installed. To insure samples would have no contact with animal derived components the initial bulk sort was performed on the single stained F-MTX population using a new flow path. Subsequent sorts were performed; the double stainedF-MTX/IgG-RPE population and the single stained IgG-RPE third population. The top 5-10% of each stained population was retained, collected, cultured and expanded in 200 nM MTX-Selection Medium, and banked at a later date.

### Cloning

### Limit Dilution Cloning 3 (LD-3) and Picking of 200 nM MTX Sorted Populations.

Using limiting dilution cloning, two sets often 384-well plates were seeded at 1.6 cell/well and 0.8 cell/well for each of the three sorts generated: yielding a total of 60, 384 well plates. Cells were only deposited into the inner wells to avoid “edge effects” (a total of 308 well/plate). On day 11 post cloning plates were randomly chosen and scored by microscope for outgrowth (See Table 2)

TABLE 2
Percent Outgrowth and corresponding plate numbers

| Stained & | 1.6 | | 0.8 | |
|---|---|---|---|---|
| sorted | cell/well | 384 | cell/well | 384 |
| population | seeding | well plate #s | seeding | well plate #s |
| f-MTX | 46 | plates 400-409 | 21 | plates 410-419 |
| IgG | 30 | plates 440-449 | 15 | plates 450-459 |
| f-MTX and | 41 | plates 420-429 | 24 | plates 430-439 |
| IgG | | | | |

Assaying, Selecting and Expanding Chosen Clones from LD-3

Colonies were picked from thirty 384-well plates from the 0.8 cell/well seeding set. Specifically, colonies were picked from plates numbered 410-419, 430-439 and 450-439. Each set represented ten plates from each of the three sorted populations. Plates were stained by Calcein AM and imaged over a two day period. After imaging, the most confluent colonies (labeled “LD-3 fast”) were manually re-suspended by a single channel pipet and transferred to 96-well plates. Each individual 96-well plate was seeded with colonies amassed from 3 individual, 384 well plates. Ten 96-well plates were thus generated. Prior to transferring colonies, the RapidPlate instrument (Caliper Sciences, Hopkinton, Mass.) was used to deposit 50 uL of 200 nM MTX-Selection Medium into each well of the ten 96-well non-tissue culture treated flat bottom plates. One week later the 0.8 cell/well 384-well plate set was stained by Calcein and imaged to commence a second round of picking. Remaining colonies from 384 wells were seeded to 96 wells as described before. These colonies were labeled “LD-3 slow”. Transferred colonies were allowed to grow in 96-well plates for one week and samples were then removed for quantification of protein by FMAT (Applied Biosystems, Foster City, Calif.). The top 384 producers (as defined by FMAT) from each of the “LD-3 fast” and “LD-3 slow” sets were then manually transferred to sixteen 24-well plates per set. The timelines for both sets differed by 1 week throughout the cloning/selecting process: with the second round picks (LD-3 slow) lagging behind the first picks (LD-3 fast). Colonies were re-suspended by pipet and the entire volume (˜100 uL) was transferred to 24-well plate wells containing 900 uL 200 nM MTX-Selection Medium. The plates were incubated for approximately one week and assayed again by FMAT. To conserve on time, the top 30 producers from each set were then transferred to 6-well plates and subcloned while simultaneously being expanded in shake flasks and adapted to suspension as pools.

### Subcloning of Limiting Dilution 3 Clones

The 30 highest producing clones from the “LD-3 fast” set and the 29 highest producing clones from the “LD-3 slow” set were each subcloned into two 384-well plates. The clones were chosen based upon FMAT results (data not shown). The RapidPlate was used to seed alternating rows of 3.2 and 1.6 cells well in one 384 well plate, and an entire 384-well plate of 0.8 cells well. Cells were seeded into every well in a volume of 50 uL of 200 nM MTX-Selection Medium+50% Conditioned-Selection Medium. Table 3 lists the 59 LD-3 clones chosen to be subcloned.

TABLE 3
LD-3 clones chosen to be subcloned.
Clone name

| LD-3 Fast | LD-3 Slow |
|---|---|
| Growers | Growers |
| 64-A12 | 73-B01 |
| 64-F10 | 73-B12 |
| 67-B08 | 73-H10 |
| 67-B10 | 74-B02 |
| 67-D05 | 74-B12 |
| 67-F05 | 74-D02 |
| 67-F09 | 74-F05 |
| 67-G04 | 74-F07 |
| 67-H12 | 75-A03 |
| 68-B01 | 75-A12 |
| 68-D07 | 75-E07 |
| 68-D11 | 76-A06 |
| 68-E03 | 76-A07 |
| 68-E11 | 76-B04 |
| 68-E12 | 76-F01 |
| 68-F07 | 77-G10 |
| 68-G02 | 77-B01 |
| 68-G07 | 77-C10 |
| 68-G08 | 77-H04 |
| 68-G11 | 77-F02 |
| 68-H01 | 78-A09 |
| 68-H02 | 78-B01 |
| 68-H11 | 78-C11 |
| 69-A07 | 78-H06 |
| 69-B08 | 79-B09 |
| 69-C05 | 79-C03 |
| 69-C09 | 79-D09 |
| 69-C11 | 80-C09 |
| 69-F03 | 80-H08 |
| 70-C07 | |

Approximately 3 weeks post seeding, clone 79-E12 (a slow grower) was also subcloned as described earlier. This clone had a difficult time adapting to culture but expressed significant levels of protein. Later the subclones were removed from the study based upon slow growth rates.

### Stability Studies on Top Amp-1 and LD-3 Subclones

Stability Studies were established for the top LD-3 subclones and the clones that were sorted. Table 4 lists the top 15 sorted subclones used for stability studies.

TABLE 4
Stability study on sorted clones

| | | | | | | Inoculum | | | |
|---|---|---|---|---|---|---|---|---|---|
| | | | | | | Req (ml) | Media | Total | |
| | Total | Viable | Viabilit [symbol] | Total/ml | Viable/ml | for | Required | Volume | New |
| Clone | cells | cells | (%) | (×10\hat{ }6 [symbol] | (×10\hat{ }6) | 0.1E+06 | (ml) | (ml) | Pass # |
| 16A030 | 305 | 260 | 85.2 | 0.29 | 0.25 | 8.15 | 20.00 | 20.00 | 1 |
| 2B02 | | | | | | | | | |
| 16A030 | 848 | 762 | 89.9 | 0.80 | 0.72 | 2.78 | 20.00 | 20.00 | 1 |
| 2C02 | | | | | | | | | |
| 16A030 | 485 | 424 | 87.4 | 0.46 | 0.40 | 5.00 | 20.00 | 20.00 | 1 |
| 2D06 | | | | | | | | | |
| 17A060 | 341 | 286 | 83.9 | 0.32 | 0.27 | 7.41 | 20.00 | 20.00 | 1 |
| 1D04 | | | | | | | | | |
| 17A06- | 1109 | 1068 | 96.3 | 1.05 | 1.01 | 1.98 | 20.00 | 20.00 | 1 |
| 02C03 | | | | | | | | | |
| 17A06- | 712 | 683 | 95.9 | 0.67 | 0.64 | 3.10 | 20.00 | 20.00 | 1 |
| 02C05 | | | | | | | | | |
| 17C11- | 541 | 517 | 95.6 | 0.51 | 0.49 | 4.10 | 20.00 | 20.00 | 1 |
| 01A01 | | | | | | | | | |
| 17C11- | 442 | 406 | 91.9 | 0.42 | 0.38 | 5.22 | 20.00 | 20.00 | 1 |
| 01D04 | | | | | | | | | |
| 17C11- | 467 | 445 | 95.3 | 0.44 | 0.42 | 4.76 | 20.00 | 20.00 | 1 |
| 02B04 | | | | | | | | | |
| 18E10- | 314 | 280 | 89.2 | 0.30 | 0.26 | 7.56 | 20.00 | 20.00 | 1 |
| 01C04 | | | | | | | | | |
| 18E10- | 243 | 211 | 86.8 | 0.23 | 0.20 | 10.04 | 20.00 | 20.00 | 1 |
| 02A01 | | | | | | | | | |
| 18E10- | 371 | 331 | 89.2 | 0.35 | 0.31 | 6.40 | 20.00 | 20.00 | 1 |
| 02C06 | | | | | | | | | |
| 29B12- | 1237 | 1171 | 94.7 | 1.17 | 1.11 | 1.81 | 20.00 | 20.00 | 1 |
| 01C02 | | | | | | | | | |
| 29B12- | 874 | 831 | 95.1 | 0.83 | 0.78 | 2.55 | 20.00 | 20.00 | 1 |
| 01C06 | | | | | | | | | |
| 29B12- | 1232 | 1174 | 95.3 | 1.16 | 1.11 | 1.80 | 20.00 | 20.00 | 1 |
| 02C03 | | | | | | | | | |

[symbol]

indicates data missing or illegible when filed

Five days later a second set of stability studies were established for 31 LD-3 subclones. Table 5 lists the 31 LD-3 subclones used for the stability study.

TABLE 5
Stability studies on LD-3 subclones

| | | | | | | | | Inoculum | | | |
|---|---|---|---|---|---|---|---|---|---|---|---|
| | | | | | | | | Req | | | |
| | | | | Total | Viable | Avg. | | (ml) for | Media | Total | New |
| | Total | Viable | Viability | cells/ml | cells/ml | diam. | Avg. | 0.1E+06 | Required | Volume | Pass |
| Clone | cells | cells | (%) | (×10\hat{ }6) | (×10\hat{ }6) | (microns) | circ. | cells/ml | (ml) | (ml) | # |
| 67D05- | 471 | 413 | 87.7 | 0.44 | 0.39 | 18.36 | 0.88 | .13 | 14.87 | 20.00 | 1 |
| 01D06 | | | | | | | | | | | |
| 67D05- | 652 | 604 | 92.6 | 0.62 | 0.57 | 18.69 | 0.88 | 3.51 | 16.49 | 20.00 | 1 |
| 01D03 | | | | | | | | | | | |
| 67D05- | 698 | 619 | 88.7 | 0.66 | 0.58 | 18.10 | 0.85 | 3.42 | 16.58 | 20.00 | 1 |
| 01C02 | | | | | | | | | | | |
| 67D05- | 557 | 486 | 87.3 | 0.53 | 0.46 | 17.75 | 0.86 | 4.36 | 15.64 | 20.00 | 1 |
| 01B01 | | | | | | | | | | | |
| 69F03- | 398 | 362 | 91.0 | 0.38 | 0.34 | 19.08 | 0.86 | 5.85 | 14.15 | 20.00 | 1 |
| 01C02 | | | | | | | | | | | |
| 69F03- | 592 | 557 | 94.1 | 0.56 | 0.53 | 16.99 | 0.88 | 3.80 | 16.20 | 20.00 | 1 |
| 01D01 | | | | | | | | | | | |
| 69F03- | 906 | 867 | 95.7 | 0.86 | 0.82 | 16.65 | 0.86 | 2.44 | 17.56 | 20.00 | 1 |
| 01A01 | | | | | | | | | | | |
| 69F03- | 963 | 920 | 95.5 | 0.91 | 0.87 | 16.28 | 0.85 | 2.30 | 17.70 | 20.00 | 1 |
| 01D06 | | | | | | | | | | | |
| 69F03- | 920 | 867 | 94.2 | 0.87 | 0.82 | 16.68 | 0.85 | 2.44 | 17.56 | 20.00 | 1 |
| 01D04 | | | | | | | | | | | |
| 73B12- | 461 | 443 | 96.1 | 0.44 | 0.42 | 15.93 | 0.90 | 4.78 | 15.22 | 20.00 | 1 |
| 01A05 | | | | | | | | | | | |
| 73B12- | 627 | 611 | 97.4 | 0.59 | 0.58 | 15.83 | 0.90 | 3.47 | 16.53 | 20.00 | 1 |
| 01A02 | | | | | | | | | | | |
| 74F07- | 501 | 476 | 95.0 | 0.47 | 0.45 | 15.56 | 0.85 | 4.45 | 15.55 | 20.00 | 1 |
| 01C02 | | | | | | | | | | | |
| 74F07- | 439 | 423 | 96.4 | 0.41 | 0.40 | 15.67 | 0.87 | 5.01 | 14.99 | 20.00 | 1 |
| 01A06 | | | | | | | | | | | |
| 75E07- | 584 | 565 | 96.7 | 0.55 | 0.53 | 15.65 | 0.87 | 3.75 | 16.25 | 20.00 | 1 |
| 01B05 | | | | | | | | | | | |
| 75E07- | 1087 | 1064 | 97.9 | 1.03 | 1.00 | 15.55 | 0.89 | 1.99 | 18.01 | 20.00 | 1 |
| 01A01 | | | | | | | | | | | |
| 76A06- | 1002 | 973 | 97.1 | 0.95 | 0.92 | 15.76 | 0.88 | 2.18 | 17.82 | 20.00 | 1 |
| 01A02 | | | | | | | | | | | |
| 76A06- | 866 | 828 | 95.6 | 0.82 | 0.78 | 16.13 | 0.88 | 2.56 | 17.44 | 20.00 | 1 |
| 01A06 | | | | | | | | | | | |
| 77F02- | 1121 | 1087 | 97.0 | 1.06 | 1.03 | 15.93 | 0.88 | 1.95 | 18.05 | 20.00 | 1 |
| 01A04 | | | | | | | | | | | |
| 77F02- | 918 | 888 | 96.7 | 0.87 | 0.84 | 15.81 | 0.86 | 2.39 | 17.61 | 20.00 | 1 |
| 01B02 | | | | | | | | | | | |
| 77G10- | 607 | 574 | 94.6 | 0.57 | 0.54 | 15.96 | 0.90 | 3.69 | 16.31 | 20.00 | 1 |
| 01C01 | | | | | | | | | | | |
| 77G10- | 633 | 602 | 95.1 | 0.60 | 0.57 | 16.07 | 0.89 | 3.52 | 16.48 | 20.00 | 1 |
| 01A04 | | | | | | | | | | | |
| 78A09- | 825 | 784 | 95.0 | 0.78 | 0.74 | 16.48 | 0.89 | 2.70 | 17.30 | 20.00 | 1 |
| 01B06 | | | | | | | | | | | |
| 78A09- | 807 | 762 | 94.4 | 0.76 | 0.72 | 16.08 | 0.88 | 2.78 | 17.22 | 20.00 | 1 |
| 01B04 | | | | | | | | | | | |
| 78B01- | 644 | 618 | 96.0 | 0.61 | 0.58 | 15.28 | 0.87 | 3.43 | 16.57 | 20.00 | 1 |
| 01A02 | | | | | | | | | | | |
| 78B01- | 801 | 788 | 98.4 | 0.76 | 0.74 | 15.57 | 0.89 | 2.69 | 17.31 | 20.00 | 1 |
| 01C05 | | | | | | | | | | | |
| 78C11- | 795 | 782 | 98.4 | 0.75 | 0.74 | 14.94 | 0.90 | 2.71 | 17.29 | 20.00 | 1 |
| 01B04 | | | | | | | | | | | |
| 78C11- | 708 | 692 | 97.7 | 0.67 | 0.65 | 15.39 | 0.89 | 3.06 | 16.94 | 20.00 | 1 |
| 01B06 | | | | | | | | | | | |
| 79B09- | 889 | 859 | 96.6 | 0.84 | 0.81 | 15.54 | 0.88 | 2.47 | 17.53 | 20.00 | 1 |
| 01A01 | | | | | | | | | | | |
| 79B09- | 1057 | 1018 | 96.3 | 1.00 | 0.96 | 15.56 | 0.88 | 2.08 | 17.92 | 20.00 | 1 |
| 01A02 | | | | | | | | | | | |
| 79D09- | 712 | 692 | 97.2 | 0.67 | 0.65 | 16.30 | 0.86 | 3.06 | 16.94 | 20.00 | 1 |
| 01D06 | | | | | | | | | | | |
| 79D09- | 700 | 681 | 97.3 | 0.66 | 0.65 | | | 3.10 | 16.90 | 20.00 | 1 |
| 01A04 | | | | | | | | | | | |

Table 6 lists the 16 stability cultures chosen to be continued: Six sorted and ten LD-3 subclones.

TABLE 6
Individual cell statistics of each of the top sixteen subclones.

| | | | | | | | | Inoculum | | Total | | Total |
|---|---|---|---|---|---|---|---|---|---|---|---|---|
| | Total | Viable | Viabilit [symbol] | Total/ml | Viabl/ml | Avg. | | Req | Media | Volume | New | Gene |
| Clone | cell | cells | (%) | (×10\hat{ } [symbol] | (×10\hat{ } [symbol] | (microns) | Avg. | (ml) | Required | (ml) | Pass | rations |
| 17A06- | 456 | 437 | 95.8 | 0.43 | 0.41 | 15.63 | 0.89 | 4.85 | 15.15 | 20.00 | 13 | 31.7 |
| 02C03 | | | | | | | | | | | | |
| 17C11- | 478 | 435 | 91.0 | 0.45 | 0.41 | 19.46 | 0.89 | 4.87 | 15.13 | 20.00 | 13 | 28.0 |
| 02B04 | | | | | | | | | | | | |
| 18E10- | 442 | 397 | 89.8 | 0.42 | 0.37 | 18.85 | 0.87 | 5.34 | 14.66 | 20.00 | 13 | 28.9 |
| 01C04 | | | | | | | | | | | | |
| 18E10- | 646 | 609 | 94.3 | 0.61 | 0.57 | 18.58 | 0.89 | 3.48 | 16.52 | 20.00 | 13 | 33.5 |
| 02C06 | | | | | | | | | | | | |
| 29B12- | 395 | 368 | 93.2 | 0.37 | 0.35 | 16.48 | 0.87 | 5.76 | 14.24 | 20.00 | 13 | 27.2 |
| 01C06 | | | | | | | | | | | | |
| 29B12- | 627 | 594 | 94.7 | 0.59 | 0.56 | 16.08 | 0.88 | 3.57 | 16.43 | 20.00 | 13 | 28.4 |
| 02C03 | | | | | | | | | | | | |
| 73B12- | 318 | 291 | 91.5 | 0.30 | 0.27 | 15.71 | 0.90 | 7.28 | 12.72 | 20.00 | 7 | 15.5 |
| 01A05 | | | | | | | | | | | | |
| 73B12- | 374 | 357 | 95.5 | 0.35 | 0.34 | 15.46 | 0.91 | 5.93 | 14.07 | 20.00 | 7 | 16.2 |
| 01A02 | | | | | | | | | | | | |
| 74F07- | 602 | 576 | 95.7 | 0.57 | 0.54 | 15.88 | 0.86 | 3.68 | 16.32 | 20.00 | 7 | 17.6 |
| 01C02 | | | | | | | | | | | | |
| 77F02- | 259 | 231 | 89.2 | 0.24 | 0.22 | 15.51 | 0.83 | 9.17 | 10.83 | 20.00 | 7 | 11.7 |
| 01B02 | | | | | | | | | | | | |
| 77G10- | 568 | 533 | 93.8 | 0.54 | 0.50 | 15.86 | 0.90 | 3.97 | 16.03 | 20.00 | 7 | 14.6 |
| 01C01 | | | | | | | | | | | | |
| 78B01- | 474 | 448 | 94.5 | 0.45 | 0.42 | 15.66 | 0.88 | 4.73 | 15.27 | 20.00 | 7 | 17.4 |
| 01A02 | | | | | | | | | | | | |
| 78C11- | 555 | 525 | 94.6 | 0.52 | 0.50 | 15.15 | 0.88 | 4.04 | 15.96 | 20.00 | 7 | 17.6 |
| 01B04 | | | | | | | | | | | | |
| 79B09- | 490 | 469 | 95.7 | 0.46 | 0.44 | 15.45 | 0.86 | 4.52 | 15.48 | 20.00 | 7 | 14.7 |
| 01A02 | | | | | | | | | | | | |
| 79D09- | 747 | 728 | 97.5 | 0.71 | 0.69 | 15.30 | 0.86 | 2.91 | 17.09 | 20.00 | 7 | 17.0 |
| 01D06 | | | | | | | | | | | | |
| 79D09- | 409 | 393 | 96.1 | 0.39 | 0.37 | 15.06 | 0.86 | 5.39 | 14.61 | 20.00 | 7 | 16.3 |
| 01A04 | | | | | | | | | | | | |

[symbol]

indicates data missing or illegible when filed

Nineteen days later 6 of the LD-3 subclones were removed from the study: 73B12-01A05, 74F07-01C02, 77G10-01C01, 78C11-01B04, 79D09-01D06 and 79D09-01A04. At this time Terminal Stability Studies were commenced on the 10 remaining subclones and completed 10 days later. Approximately two weeks later another 8 subclones were eliminated based on stability results leaving 2 remaining finalists: 18E10-01C04 and 29B12-02C03.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

What is claimed is:

1. A method for identifying a cell or cell line expressing a desired protein comprising:
i) determining in a cell or cell line that has been transfected with a vector comprising a nucleotide sequence encoding the desired protein the intracellular expression levels of elements of the vector;
ii) determining expression of the desired protein at the surface of the cell or cell line;
wherein step i) and step ii) are performed in a single assay.

2. The method according to claim 1, wherein step i) comprises determining the presence and relative abundance of the vector in a genome of the cell or cell line.

3. The method according to claim 1, wherein steps i) and ii) are performed essentially simultaneously.

4. The method according to claim 1, wherein steps i) and ii) are performed in the order recited.

5. The method according to claim 1, wherein the vector contains multiple copies of a nucleic acid sequence encoding the desired protein.

6. The method according to claim 1, wherein the vector is integrated into the genome of the cell or cell line.

7. The method according to claim 1, wherein multiple copies of the vector are integrated into the genome of the cell or cell line.

8. The method according to claim 1, wherein the vector further comprises at least one copy of a nucleic acid sequence encoding dihydrofolate reductase (DHFR).

9. The method according to claim 8, wherein the cell or cell line is deficient for dihydrofolate reductase (DHFR).

10. The method according to claim 9, further comprising treating a cell or cell line with increasing doses of methotrexate or methotrexate analog.

11. The method according to claim 8, wherein step i) comprises determining the expression level of dihydrofolate reductase (DHFR).

12. The method according to claim 8, wherein step i) comprises determining the resistance of a cell or cell line to methotrexate or methotrexate analog.

13. The method according to claim 12, wherein determining the resistance of a cell or cell line to methotrexate comprises determining the amount of methotrexate or methotrexate analog that can be taken up into a live cell while retaining viability of the cell.

14. The method according to claim 13, wherein the methotrexate or methotrexate analog comprises a fluorescent moiety.

15. The method according to claim 1, wherein step i) comprises determining a gene copy number of the vector in the genome of the cell or cell line.

16. The method according to claim 15, wherein step i) comprises exposing the cell or cell line to a labeled probe able to specifically bind to the vector.

17. The method according to claim 1, wherein step ii) comprises determining expression of the desired protein on the surface of the cell or cell line by transient expression of the protein on the cell surface.

18. The method according to claim 1, wherein step ii) comprises determining expression of the desired protein on the surface of the cell or cell line by affinity matrix retention.

19. The method according to claim 1, wherein step ii) comprises determining expression of the desired protein on the surface of the cell or cell line by gel encapsulation.

20. The method according to claim 1, wherein determining the expression of a desired protein on the surface of the cell or cell line comprises contacting the cell or cell line with a first antibody having specific binding affinity to the desired protein.

21. The method according to claim 20, wherein the first antibody comprises a fluorescent moiety.

22. The method according to claim 20, further comprising contacting the first antibody with a secondary antibody having specific binding affinity to the first antibody.

23. The method according to claim 22, wherein the secondary antibody comprises a fluorescent moiety.

24. The method according to claim 1, wherein the single assay for determining the intracellular expression levels of elements of the vector and the expression on the surface is a fluorescence based assay.

25. The method according to claim 24, wherein the fluorescence based assay is a flow cytometry assay.

26. The method according to claim 1, wherein the cell or cell line is a CHO cell or cell line.

27. The method according to claim 1, further comprising, isolating cells based on expression levels determined in steps i) and ii).

28. A cell culture comprising a population of cells enriched in the cell or cell line identified by the method of claim 27, and progeny thereof.

29. The method according to claim 1, further comprising isolating the desired protein.

30. A method for isolating a cell or cell line that has been treated with methotrexate or methotrexate analog and that expresses a desired protein comprising:
A) determining the intracellular expression levels of elements of a vector with which the cell or cell line has been transfected by determining the amount of methotrexate or methotrexate analog that can be taken up into a live cell while retaining viability of the cell;
B) determining the expression of the desired protein on the surface of the cell or cell line;
wherein the cell or cell lines are isolated based on comparing the levels of A) and B) to selected levels.

31. The method of claim 30, wherein the levels of A) and B) are at least 10% higher than the selected levels.

32. The method of claim 30, wherein the levels of A) and B) are at least 50% higher than the selected levels.

33. The method of claim 30, wherein the levels of A) and B) are at least two times higher than the selected levels.

34. An algorithm comprising determining the selected levels of A) and B) necessary for isolating the cell or cell line of claim 30, wherein the expression level of the desired protein of the isolated cell or cell line exceeds the expression level of the desired protein of an initial population of cells or cell lines from which the cell or cell line were isolated by at least about 50%.

35. An article, comprising a machine-readable medium comprising a program, embodied in the medium, for causing a machine to determine the selected levels of A) and B) necessary for isolating the cell or cell line of claim 30, wherein the expression level of the desired protein of the isolated cell or cell line exceeds the expression level of the desired protein of an initial population of cells or cell lines from which the cell or cell line were isolated by at least about 50%.

36. A business method comprising marketing or selling the cell or cell line identified in claim 1.

37. A business method comprising marketing or selling the cell or cell line isolated in claim 2.

38. A kit comprising a vector, methotrexate or a methotrexate analog and instructions for use,
wherein the vector comprises at least one copy of a nucleic acid sequence encoding dihydrofolate reductase (DHFR) and a copy of a nucleic acid encoding a desired protein.

39. The kit of claim 38, wherein the methotrexate or methotrexate analog is a fluorescent methotrexate or methotrexate analog.
